SEQUENCE LISTING (28 sequences)

SEQ ID NO: 1 - DNA, 42 bp, Artificial
  Completely artificial
tgtgtaaagagagcacgacggcaccgctggaagtgggcgcag

SEQ ID NO: 2 - protein, 14 aa, Artificial
  Completely artificial
CVKRARRHRWKWAQ

SEQ ID NO: 3 - DNA, 32 bp, Artificial
  Primer
gggcgacgggggccatggagccgctgcaatcc

SEQ ID NO: 4 - DNA, 34 bp, Artificial
  Primer
gagccgctgcaatccatggtgtgggtgaagcagc

SEQ ID NO: 5 - DNA, 32 bp, Artificial
  Primer
ccgtgctgtgggtggcacagcagcgctgcgcc

SEQ ID NO: 6 - DNA, 34 bp, Artificial
  Primer
gtgggtgaagcagcaggcctgcgccgtgagcctg

SEQ ID NO: 7 - DNA, 25 bp, Artificial
  Primer
gtgggtggcacagcaggcctgcgcc

SEQ ID NO: 8 - DNA, 30 bp, Artificial
  Primer
gcgggctctgctggcctggtggcggagccc

SEQ ID NO: 9 - DNA, 30 bp, Artificial
  Primer
ctgctgcgctggtgggcgagcccggggccc

SEQ ID NO: 10 - DNA, 39 bp, Artificial
  Primer
gcggccgctctgctggcctggtgggcgagcccggggccc

SEQ ID NO: 11 - DNA, 37 bp, Artificial
  Primer
catcaaggcagtggagcatggcagaaaatggaaaagc

SEQ ID NO: 12 - DNA, 41 bp, Artificial
  Primer
gaaacagacgttcacggggtgcatcaaggcagtggaaaatg

SEQ ID NO: 13 - DNA, 35 bp, Artificial
  Primer
cagtggaaaatggcaggcaatggaaaagccttacg

SEQ ID NO: 14 - DNA, 33 bp, Artificial
  Primer
ggcagaaaatggaagtgccttacgcttttacag

SEQ ID NO: 15 - DNA, 29 bp, Artificial
  Primer
cagtggagcatggcaggcaatggaaaagc

SEQ ID NO: 16 - DNA, 33 bp, Artificial
  Primer
ggcaggcaatggaagtgccttacgcttttacag

SEQ ID NO: 17 - DNA, 39 bp, Artificial
  Primer
gcttttacagttcactgtgtagtgagagcacgacggcac

SEQ ID NO: 18 - DNA, 34 bp, Artificial
  Primer
cagttcactgtgtaaaggcagcacgacggcaccg

SEQ ID NO: 19 - DNA, 41 bp, Artificial
  Primer
gcttttacagttcactgtgtagtggcagcacgacggcaccg

SEQ ID NO: 20 - DNA, 36 bp, Artificial
  Primer
ctgtgtaaagagagcagcagtgcaccgctggaagtg

SEQ ID NO: 21 - DNA, 32 bp, Artificial
  Primer
gagagcacgacggcacgcctggaagtgggcgc

SEQ ID NO: 22 - DNA, 31 bp, Artificial
  Primer
gacggcaccgctgggtgtgggcgcaggtgac

SEQ ID NO: 23 - DNA, 39 bp, Artificial
  Primer
gagagcacgacggcacgcctgggtgtgggcgcaggtgac

SEQ ID NO: 24 - DNA, 34 bp, Artificial
  Primer
cacgacggcacgcctgggtgtgggcgcaggtgac

SEQ ID NO: 25 - DNA, 31 bp, Artificial
  Primer
gctgtgtcacttgtgggggcagaccctgcgg

SEQ ID NO: 26 - DNA, 32 bp, Artificial
  Primer
cttgtggctgcagaccgggcgggagatgctgg

SEQ ID NO: 27 - DNA, 33 bp, Artificial
  Primer
ggctgcagaccctgcccgagatgctggagaagc

SEQ ID NO: 28 - protein, 10 aa, Artificial
  Completely artificial
KRARRHRWKW

(19) United States
(12) Patent Application Publication
(10) Pub. No.: US 2009/0081692 A1
(43) Pub. Date: Mar. 26, 2009

(54) Ceramide Kinase Loop

(76) Inventors: Markus Jaritz, Vienna (AT); Frederic Bornancin, Vienna (AT); Philipp Rovina, Vienna (AT)

Correspondence Address: NOVARTIS INSTITUTES FOR BIOMEDICAL RESEARCH, INC., 400 TECHNOLOGY SQUARE, CAMBRIDGE, MA, 02139, US

(21) Appl. No.: 12/162,857

(22) PCT Filed: Jan. 30, 2007
(86) PCT No.: PCT/EP2007/050920

§ 371 Date: Jul. 31, 2008

Related U.S. Application Data
(60) Provisional application No. 60/763,674, filed on Jan. 31, 2006.

(51) Int. Cl. C12N 5/10; C12N 15/11; C07K 7/08; C12N 15/63; C12P 21/00; G01N 33/53; C12N 1/21; C12N 1/19; C12N 1/15

(52) U.S. Cl. 435/7.1; 536/23.1; 530/327; 435/320.1; 435/252.35; 435/252.33; 435/252.31; 435/254.2; 435/254.3; 435/348; 435/365; 435/367; 435/419; 435/69.1; 435/455

(57) ABSTRACT

An isolated polynucleotide of SEQ ID NO:1, an isolated polypeptide of SEQ ID NO:2, e.g. encoded by a polynucleotide of SEQ ID NO:1, a vector comprising such polynucleotide, an expression system, comprising such polynucleotide, a host cell comprising such expression system, the use of a such polypeptide or polynucleotide as a diagnostic reagent, screening assays and methods for identifying an agonist or an antagonist of a ceramide kinase by use of such polypeptide or polynucleotide and an agonist or an antagonist of a ceramide kinase obtained by such screening and their use.

The present invention relates to a loop in the ceramide kinase sequence.

Ceramide is a sphingolipid. Sphingolipids have been considered as one of the major components of the cell membrane. Recent evidence has shown that, beyond their structural role, they can act as bioactive lipids and impact on signal transduction, in a way that is reminiscent of what is occurring with glycerophospholipids.

Physiological activity of sphingolipid metabolites include e.g. induction of apoptosis and stimulation of cell proliferation and it has been suggested that enzymes which metabolise sphingolipids are expected to participate in the induction of various diseases.

For example it has been reported
Ceramide which controls cell mechanisms has been suggested to be a regulator in the enzymatic reaction indicated above and it has been reported that ceramide works as a second messenger of inflammatory cytokines, such as TNF-α and IL-1β, and activates arachidonic pathways, such as phospholipase A₂. Ceramide thus may be considered as an exacerbating factor in inflammatory disorders;
Ceramide exacerbates the reduction of CD4⁺ T-cell accompanied by apoptosis and HIV infection of brain cells in patients infected with HIV,
in Begum N. et al, Eur. J. Biochem, 238, 214-220 (1996) and in Hotamsligil, G. S. et al, Science, 271, 665-668 (1996) it is reported that TNF-α may cause insulin resistance in type 2 diabetes mellitus as a trigger and obesity and that ceramide is involved in the downregulation of TNF-α;
Ceramide triggers septicemia caused by lipopolysaccharide;
The increase of ceramide activates sphingomyelinase in the aggregating reaction of LDL which triggers atherosclerosis lesions;
Ceramide promotes apoptosis of cancer cells in radiotherapy and chemotherapy;
Ceramide regulation is involved in drug resistance of leukemia cells: a decrease of ceramide level is associated with the chemoresistant condition in leukemia.

Also ceramide-1-phosphate (Cer-1-P), which is produced from ceramide by the action of ceramide kinase (CerK), e.g. by phosphorylation of the hydroxyl group at position 1 of various ceramide derivatives, e.g. including N-acylated-, such as N-hexanoyl-, N-octanoyl-, N-palmitoyl-D-erythro-sphingosine, shows physiological activities, e.g.
Cer-1-P produced by ceramide kinase upon calcium stimulation regulates the release of neuronal transmitters from brain synapses and modulating the action of ceramide kinase is thus expected to be of value in the treatment of various neuronal disorders, e.g. including Alzheimer's disease;
Cer-1-P is believed to inhibit various normal ceramide activities, maybe through inhibition of acid sphingomyelinase and thus Cer-1-P is expected to modulate, e.g. suppress, various disorders, e.g. inflammatory disorders, e.g. including chronic arthritis, HIV-infection, type 2 diabetes mellitus caused by insulin resistance as a trigger, obesity, septicemia and atherosclerosis; i.e. by activation of ceramide kinase it is believed that such diseases may be treated;
Cer-1-P is believed to act primarily inside the cell where it facilitates vesicle transport. It has been implicated in phagocytosis, and therefore can be expected to play an important role during inflammation processes;
the mitogenic activity of exogenously added Cer-1-P has also been shown. Therefore, this sphingolipid metabolite may be relevant to cell proliferation disorders, including but not limited to cancer and psoriasis.
Cer-1-P has been reported to mediate cytokine- and calcium ionophore-induced arachidonic release and C-1-P may directly activate cytosolic PLA2; this further evidences the possible role of Cer-1-P in inflammatory disorders;
Cer-1-P levels could also be relevant to the pathophysiology (e.g. susceptibility to retinitis pigmentosa) of the visual system.

Ceramide kinase (CerK) plays an important role in ceramide metabolism.

Surprisingly it was now found that the N-terminal domain of ceramide kinase can be unambiguously assigned to a Pleckstrin Homology (PH) domain which is required for membrane binding and conformational stability, and that a loop interconnecting the β6 and β7 strands identified is key to such processes, e.g. according to the following results. Charged amino acid residues of CerK PH domain are displayed on loops β1-β2, β3-β4, β5-β6, and β6-β7. All residues alone or in combination have been mutagenized to investigate the importance of each loop. The most critical residues are found to cluster on loop β6-β7. This loop is unique when compared to those of most known PH domains. It is highly positively charged and displays hydrophobic residues that are surrounded by charged residues. Analysis of 100 randomly generated loop models revealed low secondary structure content, but suggests at least two possible patterns: The hydrophobic residues could serve as an anchor for the loop or as a buffer between the many charged residues.

The PH domain of CerK, whose role is analyzed through through β6-β7 loop mutations effects both, localization of the enzyme and catalytic activity. Thus, the PH domain in CerK acts as an allosteric regulator. There appears to be a direct correlation between the ability of CerK to bind membrane and to be active since all mutants that are compromised in membrane binding ability also show reduced activity. The converse however seems not to be true since some inactive CerK mutants such as the ATP binding site G198D mutant, localize like the WT enzyme. As a consequence, it seems to be evident that catalytic ability is not a prerequisite for membrane binding. It can be shown that in the absence of a functional PH domain, recovery from a Triton extractable membrane component is lost.

In one aspect the present invention provides an isolated polynucleotide encoding a polypeptide of SEQ ID NO:2.

A polypeptide of SEQ ID NO:2. may be encoded by a polynucleotide of SEQ ID NO:1.

In another aspect the present invention provides an isolated polynucleotide of SEQ ID NO:1; and

An isolated polypeptide encoded by a polynucleotide of SEQ ID NO:1.

A polynucleotide provided by the present invention is herein also designated as “polynucleotide of (according to) the present invention”.

A polypeptide provided by the present invention is herein also designated as “polypeptide of (according to) the present invention”.

In another aspect the present invention provides an isolated polynucleotide of SEQ ID NO. 2.

The amino acid sequence of a polypeptide of SEQ ID NO: 2 is a part of the amino acid sequence of the sequence of ceramide kinase (CerK) as disclosed in The Journal of Biological Chemistry, Vol. 277, No. 26, pp. 23294-23300 (2002) which contains 537 amino acids in total. The loop constituted from the polypeptide of the present invention is located in the CerK sequence between amino acid positions 88 and 101 within the sequence of CerK, “Polynucleotide” as used herein, includes any polyribonucleotide or polydeoxyribonucleotide, which may be unmodified RNA or DNA, or modified RNA or DNA, including without limitation single and double stranded RNA, and RNA that is a mixture of single and double-stranded regions.

A polynucleotide according to the present invention includes a polynucleotide of SEQ ID NO:1 and allelic variants in a SEQ ID NO:1. A polynucleotide of the present invention includes a polynucleotide that hybridizes to a nucleotide sequence of a polynucleotide according to the present invention, e.g. under stringent conditions. “Stringent conditions” includes that hybridization will occur only if there is at least 80%, e.g. 90%, such as 95%, 97% or 99% identity between the nucleotide sequence of a polynucleotide according to the present invention and the corresponding polynucleotide that hybridizes.

A nucleotide sequence of a polynucleotide according to the present invention includes a sequence, which is different from SEQ ID NO:1, e.g. as a result of the redundancy (degeneracy) of the genetic code, but also encodes a polypeptide according to the present invention, e.g. or encodes a polypeptide according to the present invention of an amino acid sequence which has at least 75% identity with the amino acid sequence of the corresponding polypeptide according to the present invention with the amino acid sequence of SEQ ID NO: 2, e.g. 75% to 100%, such as 85% to 100%, e.g. (ca.) 78%, 85%, 93% or 100% identity; said identity being calculated by nₐ=xₐ-(xₐ y), wherein nₐ is the number of amino acid alterations, Xₐ is the total number of amino acids in said corresponding amino acid sequence, and y is percent identity divided by 100.

A polypeptide according to the present invention includes a polypeptide of the amino acid sequence SEQ ID NO: 2 and e.g. includes an amino acid sequence which has at least 75% identity with the amino acid sequence of SEQ ID NO: 2, e.g. 75% to 100%, such as 85% to 100%, e.g. (ca.) 78%, 85%, 93% or 100% identity; said identity being calculated as described above. An amino acid sequence having at least 75% up to 100% identity with a SEQ ID NO:2 has a comparable, preferably the same, biological activity as a polypetide according to the present invention.

A polypeptide according to the present invention may be in the form of the “mature” polypeptide, or may be part of a larger polypeptide, e.g. in the form of a fusion protein; e.g. it may be advantageous to include an additional amino acid sequence which contains secretory or leader sequences, pro-sequences, sequences which aid in purification such as multiple histidine residues, or an additional sequence for stability during recombinant production into a polypeptide of the present invention.

A polypeptide according to the present invention also includes a polypeptide fragment of a polypeptide according to the present invention. Such polypeptide fragment is meant to be a polypeptide having an amino acid sequence that entirely is the same in part, but not in all, of the amino acid sequence of a polypeptide of the present invention. Such polypeptide fragment may be “free-standing,” or may be part of a larger polypeptide of which such polypeptide fragment form a part or region, most preferably as a single continuous region. Preferably such polypeptide fragment retains the biological activity of a polypeptide according to the present invention.

Variants of defined polypeptide (fragment) sequences according to the present invention also form part of the present invention. Preferred variants are those that vary from the referents by conservative amino acid substitutions, e.g. those that substitute a residue with another of like characteristics. Typically such substitutions are among Ala and Val, and among the basic residues Lys and Arg. Particularly preferred are variants in which 1 to 2 amino acids are substituted, deleted, or added in any combination.

A polypeptide according to the present invention includes isolated a naturally occurring polypeptide of the present invention, a recombinantly produced polypeptide, a synthetically produced polypeptide, or a polypeptide produced by a combination of these methods. A polypeptide or fragment thereof of the present invention may be produced as appropriate, e.g. according to a method as conventional, or as described herein. “Isolated”, if not otherwise specified herein includes the meaning “separated from the coexisting material”, e.g. “altered by the hand of man” from the natural state.

A polynucleotide according to the present invention may be used for the recombinant production of a corresponding polypeptide according to the present invention. If a polynucleotide according to the present invention is used for the recombinant production of a polypeptide of the present invention, the polynucleotide sequence may include the coding sequence for the mature polypeptide by itself; the coding sequence for the mature polypeptide in reading frame with other coding sequences, such as those encoding a leader or secretory sequence, a pre- or pro- or prepro-protein sequence, or other fusion peptide portions. For example, a marker sequence which facilitates purification of a fused polypeptide, can be encoded. The marker sequence may be an appropriate marker sequence, e.g. including conventional marker sequences, e.g. a hexa-histidine peptide, as provided in the pQE vector (Qiagen, Inc.) and described in Gentz et al., Proc Natl Acad Sci USA (1989) 86:821-824, or an HA tag. Any polynucleotide according to the present invention may also contain non-coding 5′ and 3′ sequences, such as transcribed, non-translated sequences, splicing and polyadenylation signals, ribosome binding sites and sequences that stabilize mRNA.

A nucleotide sequence which is identical or sufficiently identical to the nucleotide sequence of a polynucleotide according to the present invention, may be used as an hybridization probe for cDNA and genomic DNA, to isolate full-length cDNAs and genomic clones encoding ceramide kinase; and e.g. to isolate cDNA and genomic clones of other polynucleotides (including polynucleotides encoding homologs and orthologs from species other than human) that have a high sequence similarity to a polynucleotide according to the present invention. Any appropriate hybridization technique may be used, e.g. comprising the steps of screening an appropriate library under stringent hybridization conditions with a labeled probe having the corresponding polynucleotide sequence or that of a splice variant thereof or a fragment thereof, and isolating full-length cDNA and genomic clones containing said polynucleotide sequence. Hybridization techniques, e.g. stringent, are well known. Stringent hybridization conditions e.g. are as defined above, e.g. or, alternatively, conditions under overnight incubation at around 40° C. in an appropriate solution, e.g. comprising a solution comprising formamide, SSC, sodium phosphate, Denhardt's, dextran, salmon sperm DNA, e.g. comprising 50% formamide, 5×SSC (150 mM NaCl, 15 mM trisodium citrate), 50 mM sodium phosphate (pH7.6), 5×Denhardt's solution, 10% dextran sulfate, and 20 microgram/ml denatured, sheared salmon sperm DNA, followed by washing the filters in 0.1×SSC at about 65° C. If a polynucleotide encoding a ceramide kinase as described herein is isolated by such procedure, a polynucleotide according to the present invention may be obtained therefrom by use of an appropriate method, e.g. such as conventional.

In another aspect the present invention provides a vector comprising a polynucleotide of the present invention.

A vector comprising a polynucleotide according to the present invention may be produced as appropriate, e.g. according to a method as conventional, e.g. using an appropriate vector. An appropriate vector may be provided as appropriate, e.g. according to a method as conventional. A vector comprising a polynucleotide of the present invention may be useful to obtain an expression system which is able to produce a polypeptide encoded by a polynucleotide according to the present invention recombinantly, e.g. in a host cell, such as in a compatible host cell. E.g. for recombinant production of a polypeptide according to the present invention a host cell may be genetically engineered, e.g. by use of a vector comprising a polynucleotide according to the present invention, to incorporate into the host cell an expression system, e.g. or a part thereof, for expressing a polypeptide (fragment) of the present invention. Cell-free translation systems may also be used to produce a polynucleotide according to the present invention, e.g. using RNAs derived from an DNA construct according to the present invention; e.g. according to a method as conventional.

In another aspect the present invention provides an expression system, comprising a polynucleotide of the present invention, e.g. comprising an DNA or RNA molecule isolated from the natural environment, e.g. comprising an pre-isolated polynucleotide according to the present invention, wherein said expression system or part thereof is capable of producing a polypeptide of the present invention, when said expression system or part thereof is present in a compatible host cell.

In another aspect the present invention provides
an, e.g. isolated, host cell comprising an expression system according to the present invention;
a process for producing a polypeptide according to the present invention comprising culturing a host cell comprising an expression system according to the present invention under conditions sufficient for the production of a polypeptide of the present invention in the culture and recovering a polypeptide of the present invention from the culture;
a process for the production of a recombinant host cell which produces a polypeptide according to the present invention comprising transforming or transfecting a host cell with the expression system according to the present invention such that the host cell, under appropriate culture conditions, produces a polypeptide according to the present invention; and
a recombinant host cell produced by transforming or transfecting a host cell with the expression system according to the present invention such that the host cell, under appropriate culture conditions, produces a polypeptide according to the present invention.

For recombinant production, host cells may be genetically engineered to incorporate expression systems or portions thereof for a gene according to the present invention. Introduction of polynucleotides into host cells may be effected as appropriate, e.g. according to a method as conventional, e.g. according to Davis et al., BASIC METHODS IN MOLECULAR BIOLOGY (1986); Sambrook et al., MOLECULAR CLONING: A LABORATORY MANUAL, 2nd Ed., Cold Spring Harbor Laboratory Press, Cold Spring Harbor, N.Y. (1989) such as calcium phosphate transfection, DEAE-dextran mediated transfection, transvection, microinjection, cationic lipid-mediated transfection, electroporation, transduction, scrape loading, ballistic introduction or infection. Host cells may be easily found. Examples of appropriate host cells include e.g. bacterial cells, such as streptococci, staphylococci, E. coli, Streptomyces and Bacillus subtilis cells; fungal cells, such as yeast cells and Aspergillus cells; insect cells such as Drosophila S2 and Spodoptera Sf9 cells; isolated animal cells such as CHO, COS, HeLa, C127, CCL39, 3T3, BHK, HEK 293 and Bowes melanoma cells; and plant cells.

Appropriate expression systems include e.g. chromosomal, episomal and virus-derived systems, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids. An expression systems may contain control regions that regulate as well as engender expression. Generally, any system or vector suitable to maintain, propagate or express polynucleotides to produce a polypeptide in a host may be used. The appropriate nucleotide sequence may be inserted into an expression system as appropriate, e.g. according to a method as conventional, e.g. according to Sambrook et al., MOLECULAR CLONING A LABORATORY MANUAL (supra).

A polypeptide according to the present invention may be recovered and purified from recombinant cell cultures as appropriate, e.g. according to a method as conventional, e.g. including detergent extraction, ultracentrifugation, ammonium sulfate or ethanol precipitation, acid extraction, anion or cation exchange chromatography, phosphocellulose chromatography, hydrophobic interaction chromatography, affinity chromatography, hydroxylapatite chromatography, lectin chromatography, e.g. high performance liquid chromatography. If a polypeptide according to the present invention is denatured during isolation and or purification, regeneration of the active conformation, e.g. refolding of a denaturated polypeptide of the present invention, may be carried out as appropriate, e.g. according to a method as conventional.

A polynucleotide of the present invention or a polypeptide of the present invention may be used as a research reagent and as a tool for the discovery of treatments and diagnostics to animal and human diseases.

In another aspect the present invention provides the use of a polynucleotide or a polypeptide of the present invention as a diagnostic reagent.

The present invention also provides the use of a polynucleotide or a polypeptide according to the present invention as a diagnostic reagent. Detection of a mutated form of a polynucleotide (polypeptide) according to the present invention associated with a dysfunction will provide a diagnostic tool, e.g. in a diagnostic assay, that may add to or define a diagnosis of a disease or susceptibility to a disease which results from under-expression, over-expression or altered expression of the corresponding mutant version of a polynucleotide of the present invention. Individuals carrying mutations in the corresponding positions of the polynucleotide encoding the CerK sequence between amino acid positions 88 and 101 in a mutated form may be detected at the DNA level e.g. analogously to a method as conventional. Nucleic acids for diagnosis may be obtained from a subject's cells, such as from blood, urine, saliva, tissue biopsy or autopsy material. The genomic DNA may be used directly for detection or may be amplified enzymatically by using PCR or other amplification techniques prior to analysis. RNA or cDNA may also be used in the analysis similarity. Deletions and insertions may be detected by a change in size of the amplified product in comparison to the normal genotype. Point mutations may be identified by hybridizing amplified DNA to labeled nucleotide sequences of the present invention. Perfectly matched sequences may be distinguished from mismatched duplexes by RNase digestion or by differences in melting temperatures. DNA sequence differences may also be detected by alterations in electrophoretic mobility of DNA fragments in gels, with or without denaturing agents, or by direct DNA sequencing, e.g. according to Myers et al, Science (1985) 230:1242. Sequence changes at specific locations may also be revealed by nuclease protection assays, such as RNase and S1 protection or the chemical cleavage method, e.g. according to Cotton et al, Proc Natl Acad Sci USA (1985) 85: 4397-4401. An array of oligonucleotides probes comprising the nucleotide sequence of the present invention or fragments thereof may be constructed to conduct efficient screening of e.g. genetic mutations. Array technology methods may e.g. be used to address a variety of questions in molecular genetics including gene expression, genetic linkage, and genetic variability, e.g. according to M. Chee et al, Science, Vol 274, pp 610-613, 1996.

A polypeptide of the present invention provides in several aspects a target for a pharmaceutical against various disorders, such as disorders which are mediated by CerK activity.

Disorders which are mediated by CerK activity and which are prone to be successfully treated with a modulator of CerK, include e.g. disorders wherein the activity of CerK plays a causal or contributory role, such as disorders associated with the binding of ceramide kinase to ceramide, e.g. associated with delaying or accelerating phosphorylation of ceramide by ceramide kinase.

Disorders which are prone to be mediated by CerK activity e.g. include
Disorders Associated with Inflammation
e.g. including (chronic) inflammatory disorders, disorders related with the inflammation of the bronchi, e.g. including bronchitis, cervix, e.g. including cervicitis, conjunctiva, e.g. conjunctivitis, esophagus, e.g. esophagitis, heart muscle, e.g. myocarditis, rectum, e.g. proctitis, sclera, e.g. scleritis, gums, involving bone, pulmonary inflammation (alveolitis), airways, e.g. asthma, such as bronchial asthma, acute respiratory distress syndrome (ARDS), inflammatory skin disorders such as contact hypersensitivity, atopic dermatitis; fibrotic disease (e.g., pulmonary fibrosis), encephilitis, inflammatory osteolysis,
Disorders Associated with Conditions of the Immune System,
immune, such as autoimmune disorders e.g. including Graves' disease, Hashimoto's disease (chronic thyroiditis), multiple sclerosis, rheumatoid arthritis, arthritis, gout, osteoarthritis, scleroderma, lupus syndromes, systemic lupus erytomatosis, Sjogren's syndrome, psoriasis, inflammatory bowel disease, including Crohn's disease, colitis, e.g. ulcerative colitis; sepsis, septic shock, autoimmune hemolytic anemia (AHA), autoantibody triggered urticaria, pemphigus, nephritis, glomerulonephritis, Goodpastur syndrome, ankylosing spondylitis, Reiter's syndrome, polymyositis, dermatomyositis, cytokine-mediated toxicity, interleukin-2 toxicity, alopecia areata, uveitis, lichen planus, bullous pemphigoid, myasthenia gravis, type I diabetes mellitus, immune-mediated infertility such as premature ovarian failure, polyglandular failure, hypothyroidism, pemphigus vulgaris, pemphigus I-oliaceus, paraneoplastic pemphigus, autoimnune hepatitis including that associated with hepatitis B virus (HBV) and hepatitis C virus (HCV), Addison's disease, autoimmune skin diseases, such as psoriasis, dermatitis herpetiformis, epidermolysis bullosa, linear IgA bullous dermatosis, epidermolysis bullosa acquisita, chronic bullous disease of childhood, pernicious anemia, hemolytic anemia, vitiligo, type I, type II and type III autoimmune polyglandular syndromes, Autoimmune Hypoparathyroidism, Autoimmune Hypophysitis, Autoimmune Oophoritis, Autoimmune Orchitis, pemphigoid gestationis, cicatricial pemphigoid, mixed essential cryoglobulinemia, immune thrombocytopenic purpura, Goodpasture's syndrome, autoimmune neutropenia, Eaton-Lambert myasthenic syndrome, stiff-man syndrome, encephalomyelitis, acute disseminated encephalomyelitis, Guillain-Barre syndrome, cerebellar degeneration, retinopathy, primary biliary sclerosis, sclerosing cholangitis autoimmune hepatitis, gluten-sensitive enteropathy, reactive arthritides, polymyositis/dermatomyositis, mixed connective tissue disease, Bechet's syndrome, polyarteritis nodosa allergic anguitis and granulomatosis (Churg-Strauss disease), polyangiitis overlap syndrome (hypersensitivity) vasculitis, Wegener's granulomatosis, temporal arteritis Kawasaki's disease, sarcoidosis, cryopathies, Celiac disease,
Disorders Associated with Cytokine-Mediated Toxicity,
e.g. including interleukin-2 toxicity,
Disorders Associated with the Bone,
e.g. including osteoporosis, osteoarthritis,
Disorders Associated with the Brain and the Nerves,
neurodegenerative disorders, e.g. including disorders of the central nervous system as well as disorders of the peripheral nervous system, e.g. CNS disorders including central nervous infections, brain injuries, cerebrovascular disorders and their consequences, Parkinson's disease, corticobasal degeneration, motor neuron disease, dementia including ALS, multiple sclerosis, traumatic disorders, including trauma and inflammatory consequences of trauma, traumatic brain injury, stroke, post-stroke, post-traumatic brain injury,
small-vessel cerebrovascular disease, eating disorders; further dementias, e.g. including Alzheimer's disease, vascular dementia, dementia with Lewy-bodies, frontotemporal dementia and Parkinsonism linked to chromosome 17, frontotemporal dementias, including Pick's disease, progressive nuclear palsy, corticobasal degeneration, Huntington's disease, thalamic degeneration, Creutzfeld Jakob dementia, HIV dementia, schizophrenia with dementia, Korsakoff's psychosis,
cognitive-related disorders, such as mild cognitive impairment, age associated memory impairment, age-related cognitive decline, vascular cognitive impairment, attention deficit disorders, attention deficit hyperactivity disorders, and memory disturbances in children with learning disabilities; conditions associated with the hypothalamic-pituitary-adrenal axis,
neuronal disorders, e.g, including neuronal migration disorders, hypotonia (reduced muscle tone), muscle weakness, seizures, developmental delay (physical or mental development difficulty), mental retardation, growth failure, feeding difficulties, lymphedema, microcephaly, symptoms affecting the head and the brain, motor dysfunction;
Disorders Associated with the Eye,
e.g. including uveoretinitis, vitreoretinopathy, corneal disease, iritis, iridocyclitis, cataracts, uveitis, diabetic retinopathy, retinitis pigmentosa, conjunctivitis, keratitis,
Disorders Associated with the Gastrointestinal Tract
e.g. including colitis, inflammatory bowel disease, Crohn's disease, ulcerative colitis, peptic ulceration, gastritis, oseophagitis,
Disorders Associated with the Heart and Vascular Conditions
e.g. including cardiovascular disorders, e.g. including cardiac failure, cardiac infarction, cardiac hypertrophy, heart failure, e.g. including all forms of heart pumping failures such as high-output and low-output, acute and chronic, right sided or left-sided, systolic or diastolic, independent of the underlying cause; myocardial infarction (MI), MI prophylaxis (primary and secondary prevention), acute treatment of MI, prevention of complications; heart disorders, proliferative vascular disorders, vasculitides, polyarteritis nodosa, inflammatory consequences of ischemia, ischemic heart disease, myocardial infarction, stroke, peripheral vascular disease, pulmonary hypertension,
ischemic disorders, e.g. including myocardial ischemia, e.g. stable angina, unstable angina, angina pectoris, bronchitis; asymptomatic arrhythmias such as all forms of atrial and ventricular tachyarrhythmias, atrial tachycardia, atrial flutter, atrial fibrillation, atrioventricular reentrant tachycardia, preexitation syndrome, ventricular tachycardia, ventricular flutter, ventricular fibrillation, bradycardic forms of arrhythmias; arrhythmia, chronic obstructive pulmonary disease,
hypertension, such as systolic or diastolic high blood pressure, e.g essential and secondary hypertension, e.g. including hypertensive vascular disorders, such as primary as well as all kinds of secondary arterial hypertension, renal, endocrine, neurogenic and others; peripheral vascular disorders in which arterial and/or venous flow is reduced resulting in an imbalance between blood supply and tissue oxygen demand, e.g. including artherosclerosis, chronic peripheral arterial occlusive disease (PAOD), acute arterial thrombosis and embolism, inflammatory vascular disorders, Raynaud's phenomenon and venous disorders; atherosclerosis, a disease in which the vessel wall is remodeled, e.g. including accumulation of cells, both smooth muscle cells and monocyte/macrophage inflammatory cells, in the intima of the vessel wall;
hypotension,
Disorders Associated with the Liver and the Kidneys,
e.g. including renal disorders, kidney disorders, e.g. acute kidney failure, acute renal disease, liver disorders, e.g. cirrhosis, hepatitis, liver failure, cholestasis, acute/chronic hepatitis, sclerosing cholangitis, primary biliary cirrhosis, acute/chronic interstitial/glomerulonephritis, granulomatous diseases,
Disorders Associated with Stomach or Pancreas Conditions
e.g. including stomach disorders, e.g. gastric ulcer, gastrointestinal ulcer, pancreatic disorders, pancreatic fatigue,
Disorders Associated with the Respiratory Tract and Lung
e.g. including pulmonary disorders, chronic pulmonary disease, acute (adult) respiratory distress syndrome (ARDS), asthma, asthma bronchitis, bronchiectasis, diffuse interstitial lung disorders, pneumoconioses, fibrosing aveolitis, lung fibrosis,
Disorders Associated with Skin and Connective Tissue Conditions
e.g. including eczema, atopic dermatitis, contact dermatitis, psoriasis, acne, dermatomyositis, Sjögren's syndrome, Churg-Struass syndrome, sunburn, skin cancer, wound healing, urticaria, toxic epidermal necrolysis,
Disorders Associated with Allergic Conditions,
e.g. including delayed-type hypersensitivity, allergic conjunctivitis, drug allergies, rhinitis, allergic rhinitis, vasculitis, contact dermatitis;
Disorders Associated with Angiogenesis,
e.g. including insufficient ability to recruit blood supply, disorders characterised by odified angiogenesis, tumor associated angiogenesis,
Disorders Associated with Cancer and Cell Overproliferation,
e.g. including premalignant conditions, hyperproliferative disorders, cancers whether primary or metastatic, cervical and metastatic cancer, cancer originating from uncontrolled cellular proliferation, solid tumors, such as such as described in WO02066019, including nonsmall cell lung cancer, cervical cancer; tumor growth, lymphoma, B-cell or T-cell lymphoma, benign tumors, benign dysproliferative disorders, renal carcinoma, esophageal cancer, stomach cancer, renal carcinoma, bladder cancer, breast cancer, colon cancer, lung cancer, melanoma, nasopharyngeal cancer, osteocarcinoma, ovarian cancer, uterine cancer; prostate cancer, skin cancer, leukemia, tumor neovascularization, angiomas, myelodysplastic disorders, unresponsiveness to normal death-inducing signals (immortalization), increased cellular motility and invasiveness, genetic instability, dysregulated gene expression, (neuro)endocrine cancer (carcinoids), blood cancer, lymphocytic leukemias, neuroblastoma; soft tissue cancer, prevention of metastasis,
Disorders Associated with Diabetic Conditions,
e.g. including diabetes (type I diabetes, type II diabetes), diabetic retiropathy, insulin-dependent diabetes, diabetes mellitus, gestational diabetes), insulin hyposecretion, obesity;
Disorders Associated with Endiometriosis, Testicular Dysfunctions,
Disorders Associated with Infectious Disorders, e.g. with Chronic Infectious Conditions,
e.g. including bacterial disorders, otitis media, Lyme disease, thryoditis, viral disorders, parasitic disorders, fungal disorders, malaria, e.g. malaria anemia, sepsis, severe sepsis, septic shock, e.g. endotoxin-induced septic shock, exotoxin-induced toxic shock, infective (true septic) shock, septic shock caused by Gram-negative bacteria, pelvic inflammatory disease, AIDS, enteritis, pneumonia; meningitis, encephalitis,
Disorders Associated with Myasthenia Gravis,
Disorders Associated with Nephritis,
e.g. including glomerulonephritis, interstitial nephritis, Wegener's granulomatosis, fibrosis,
Disorders Associated with Pain,
e.g. associated with CNS disorders, such as multiple sclerosis, spinal cord injury, sciatica, failed back surgery syndrome, traumatic brain injury, epilepsy, Parkinson's disease, post-stroke, and vascular lesions in the brain and spinal cord (e.g., infarct, hemorrhage, vascular malformation);
non-central neuropathic pain, e.g. including that associated with post mastectomy pain, phantom feeling, reflex sympathetic dystrophy (RSD), trigeminal neuralgiaradioculopathy, post-surgical pain, HIV/AIDS related pain, cancer pain, metabolic neuropathies (e.g., diabetic neuropathy, vasculitic neuropathy secondary to connective tissue disease), paraneoplastic polyneuropathy associated, for example, with carcinoma of lung, or leukemia, or lymphoma, or carcinoma of prostate, colon or stomach, trigeminal neuralgia, cranial neuralgias, and post-herpetic neuralgia;
pain associated with peripheral nerve damage, central pain (i.e. due to cerebral ischemia) and various chronic pain i.e., lumbago, back pain (low back pain), inflammatory and/or rheumatic pain;
headache pain (for example, migraine with aura, migraine without aura, and other migraine disorders), episodic and chronic tension-type headache, tension-type like headache, cluster headache, and chronic paroxysmal hemicrania;
visceral pain such as pancreatits, intestinal cystitis, dysmenorrhea, irritable Bowel syndrome, Crohn's disease, biliary colic, ureteral colic, myocardial infarction and pain syndromes of the pelvic cavity, e.g., vulvodynia, orchialgia, urethral syndrome 15 and protatodynia;
acute pain, for example postoperative pain, and pain after trauma;
Disorders Associated with Rheumatic Disorders,
e.g. including arthritis, rheumatoid arthritis, osteoarthritis, psoriatic arthritis, crystal arthropathies, gout, pseudogout, calcium pyrophosphate deposition disease, lupus syndromes, systemic lupus erythematosus, sclerosis, sclerodema, multiple sclerosis, artherosclerosis, arteriosclerosis, spondyloarthropathies, systemic sclerosis, reactive arthritis, Reiter's syndrome, ankylosing spondylitis, polymyositis,
Disorders Associated with Sarcoidosis,
Disorders Associated with Transplantation,
e.g. including transplant rejection crisis and other disorders following transplantation, such as organ or tissue (xeno)transplant rejection, e.g. for the treatment of recipients of e.g. heart, lung, combined heart-lung, liver, kidney, pancreatic, skin, corneal transplants, graft versus host disease, such as following bone marrow transplantation, ischemic reperfusion injury.

Disorders as used herein include diseases.

Disorders mediated by CerK activity which are prone to be successfully treated with CerK agonists, such as compounds of the present invention, preferably include disorders associated with inflammation, disorders associated with conditions of the immune system, e.g. autoimmune disorders, such as rheumatoid arthritis, inflammatory bowel disease, systemic lupus erytomatosis, multiple sclerosis, disorders associated with allergic conditions, disorders associated with cancer and cell overproliferation, disorders associated with transplantation, disorders associated with diabetic conditions, e.g. type 2 diabetes mellitus, e.g. caused by insulin resistance as a trigger, obesity; disorders associated with infectious disorders, e.g. HIV-infection, disorders associated with the brain and the nerves (neuronal disorders), disorders associated with pain, disorders associated with the eye, e.g. retinitis pigmentosa;

more preferably rheumatoid arthritis, inflammatory bowel disease, systemic lupus erytomatosis, multiple sclerosis, transplant rejection crisis, psoriasis, cancer and AIDS, more preferably rheumatoid arthritis, inflammatory bowel disease, systemic lupus erytomatosis, multiple sclerosis, psoriasis.

Treatment as used herein includes treatment and prophylaxis (prevention).

Disorders as used herein include diseases.

A diagnostic assay offers a process for diagnosing or determining a susceptibility to disorders mediated by the action of CerK.

A disorder may be diagnosed e.g. analogously to a method as conventional, e.g. by determining from a sample derived from a subject
a) a mutation between amino acid positions 88 and 101 position of a CerK as described herein, and
b) an abnormally decreased or increased level of a
   (i) CerK protein,
   (ii) secondary metabolite of such CerK protein, such as ceramide-1-phosphate, or a related lipid metabolite-phosphate, and/or
   (iii) mRNA encoding CerK.

A mutation between amino acid positions 88 and 101 position of a CerK may be determined by isolating such CerK and determining the amino acid sequence between amino acid positions 88 and 101 of CerK. Decreased or increased expression levels can be determined at the RNA level, e.g. according to a method as conventional for the quantitation of polynucleotides, such as, for example, PCR, RT-PCR, RNase protection, Northern blotting and other hybridization methods. Assay techniques that may be used to determine levels of a protein, such as CerK protein comprising a mutation in the amino acid sequence between amino acid positions 88 and 101, or to determine secondary metabolites of such ceramide kinase protein in a sample derived from a host may be carried out as appropriate, e.g. analogously to a method as conventional. Such assay techniques include radioimmunoassays, competitive-binding assays, Western Blot analysis, ELISA and methods for detecting the amount of secondary metabolites, e.g. ceramide-1-phosphate, e.g. including fluorescent methods, mass spectrometry and chromatography.

In another aspect the present invention provides a diagnostic kit for a disorder or susceptibility to a disorder, such as described above, for the case that a mutation in the amino acid sequence between amino acid positions 88 and 101 of a ceramide kinase as described herein is present, comprising as a main component
a) ceramide kinase polynucleotide, or
b) a nucleotide sequence complementary to that of a), or
c) a ceramide kinase protein, or
d) an antibody against a ceramide kinase protein, and
e) means for detecting a mutation in the amino acid sequence between amino acid positions 88 and 101 of CerK, if present,
   e.g. any such kit, (a), (b), (c) and/or (d) may comprise a substantial component, e.g. including
   an appropriate environment of a sample,
   appropriate means to determine the effect of any of (a), (b), (c) or (d), in a sample to be tested.

The amino acid sequence between amino acid positions 88 and 101 of a ceramide kinase as described herein may be responsible for many biological functions, including many pathologies, e.g. disorders such as described above. Accordingly, it is desirous to find compounds/drugs which either stimulate (agonists)
ceramide kinase, e.g. to produce secondary metabolites,
the expression of a polynucleotide encoding ceramide kinase, e.g. to stimulate ceramide kinase expression,
   or which reduce or inhibit (antagonists)
the action of ceramide kinase, e.g. to reduce or inhibit the production of secondary metabolites
the expression of a polynucleotide encoding ceramide kinase.

A polypeptide of the present invention or functional mimetics thereof, e.g. according to Coligan et al, Current Protocols in Immunology 1 (2):Chapter 5 (1991), may thus be used to assess the binding of agonists or antagonists to a receptor part of the polypeptide of the present invention, e.g. in cells, cell-free preparations, chemical libraries, and natural product mixtures, e.g. in a screening assay.

Such agonists and antagonists (modulators) may be used for the treatment of disorders as described above.

Screening procedures may involve the production of appropriate cells in which a polypeptide of the present invention is expressed. Appropriate cells include cells e.g. from mammals, yeast, Drosophila. Cells expressing a polypeptide (or cell membranes containing the expressed polypeptide of the present invention) may be contacted with a candidate compound (potential modulator) to observe binding, or stimulation or inhibition of a functional response.

A screening assay may be used to test the binding of a candidate compound to a polypeptide of the present invention wherein binding may be detected by means of a label directly or indirectly associated with the candidate compound or in an assay involving competition with a labeled competitor.

Modulators of activation may be assayed in the presence and in the absence of a known (ant)agonist.

A functional screening assay may comprise the steps of mixing a candidate compound from which binding to a polypeptide of the present invention has been determined with a solution containing ceramide kinase, to form a mixture, determining activity of ceramide kinase in the mixture, and comparing the activity of the mixture with the activity of a standard. A ceramide kinase (cDNA), a polypeptide of ceramide kinase, and antibodies to ceramide kinase may also be used to provide a screening assay for detecting the effect of candidate compounds from which binding to a polypeptide of the present invention has been determined, on the production of said ceramide kinase (mRNA) and said ceramid kinase polypeptide in cells. For example, an ELISA may be constructed for determining cell associated levels of said polypeptide, e.g. using monoclonal and polyclonal antibodies according to a method as conventional, and that ELISA may be used to discover agents (modulators) from which binding to a polypeptide of the present invention has been determined, which may increase or inhibit the production or the activity of ceramide kinase from suitably manipulated cells or tissues. An assay for screening may be conducted, e.g. according to a method as conventional.

Examples of potential (ant)agonists which may bind to a polypeptide of the present invention include e.g. including oligopeptides, polypeptides, protein, antibodies, mimetics, small molecules, e.g. low molecular weight compounds (LMW's).

Thus in another aspect, the present invention provides a screening assay for identifying an agonist or an antagonist of a ceramide kinase as described herein which assay comprises as a main component
a) a polypeptide of the present invention, or
b) a recombinant cell expressing a polypeptide of a), or
c) a cell membrane expressing a polypeptide according to a), and
d) means for determining a binding effect of a candidate compound with a polypeptide of the present invention,
   e.g. and means for a contact with a candidate compound; e.g. and means for determining the effect of the candidate compound on any of a), b) or c) and d);
   e.g. determining whether in the presence of the candidate compound there is a decrease or increase in the production and or the biological activity of a ceramide kinase as described herein;
   e.g. by comparison of the activity of any of a), b) or c) and d) in the presence and in the absence of said candidate compound;
   and in another aspect

The use of a polypeptide of the present invention in such screening assay for identifying an (ant)agonist.

In another aspect the present invention provides

A method of identfying an agonist or antagonist which increases or decreases the production and/or the biological activity of ceramide kinase, which comprises
A) contacting
A2) a polypeptide of the present invention, or
A2) a recombinant cell expressing a polypeptide of A1), or
A3) a cell membrane expressing a polypeptide of A1), with a candidate compound,
B) determining a binding effect of a candidate compound with the polypeptide of any of A1), A2) or A3),
C) determining the effect of a candidate compound from which a binding effect has been determined in step B) on any of A1), A2) or A3) on;
C1) ceramide kinase protein, or
C2) a recombinant cell expressing ceramide kinase protein, or
C3) a cell membrane expressing ceramide kinase protein, or
C4) an antibody to a polypeptide of C1),
   e.g. determining whether in the presence of the candidate compound there is a decrease or increase in the production and or the biological activity of ceramide kinase;
   e.g. by comparison of the activity of any of C1), C2), C3) or C4) in the presence and in the absence of said candidate compound, and
D) choosing an agonist or antagonist determined in step C),
   e.g, choosing an appropriate candidate compound from which an agonist/antagonist effect is positively determined in step C);
   and in another aspect

The use of a polypeptide of the present invention in such method for the identifying an (ant)agonist.

An (ant)agonist (modulator) is a candidate compound from which an effect on any of C1), C2), C3) or C4) has been found in a screening assay or in a method for identifying (ant)agonists as described above. An (ant)agonist may decrease or increase the production and or the biological activity of ceramide kinase. Such (ant)agonist is also designated herein as an (ant)agonist of (according to) the present invention.

In another aspect the present invention provides an agonist or an antagonist of a polypeptide of ceramide kinase protein, which is characterized in that said agonist or antagonist can be provided by a method for identfying an agonist or antagonist of the present invention.

An (ant)agonist of a polypeptide according to the present invention may be used in the treatment of disorders, e.g. such as described herein. An (ant)agonist of a polypeptide according to the present invention may be useful as a pharmaceutical.

In another aspect the present invention provides an agonist or an antagonist of a polypeptide of ceramide kinase for use as a pharmaceutical, e.g. for the treatment of disorders, such as described herein.

An (ant)antagonist of ceramide kinase protein of the present invention may be administered in the form of a pharmaceutical composition.

In another aspect the present invention provides
A pharmaceutical composition comprising an agonist or an antagonist of the present invention as an active ingredient in combination with pharmaceutically acceptable excipient(s), e.g. which is characterized in that said antagonist or agonist can be provided by a method of identfying an agonist or antagonist of the present invention.

Such pharmaceutical composition may be produced as appropriate, e.g. according, e.g. analogously, to a method as conventional, e.g. by mixing an (ant)agonist provided by the method steps A), B) and C) with excipients, e.g. and further processing the mixture obtained, to obtain a pharmaceutical composition for appropriate administration.

In a further aspect the present invention provides a method of treating disorders mediated by ceramide kinase activity,

comprising administering a therapeutically effective amount of an agonist or antagonist of the present invention, e.g. which can be provided by the method steps A), B), C) and D) as described above, e.g. in combination with pharmaceutically acceptable excipient(s);
e.g. in the form of a pharmaceutical composition;
to a subject in need of such treatment.

For such treatment, the appropriate dosage will, of course, vary depending upon, for example, the chemical nature and the pharmacokinetic data of a compound of the present invention used, the individual host, the mode of administration and the nature and severity of the conditions being treated. However, in general, for satisfactory results in larger mammals, for example humans, an indicated daily dosage includes a range
from about 0.0001 g to about 1.5 g, such as 0.001 g to 1.5 g;
from about 0.001 mg/kg body weight to about 20 mg/kg body weight, such as 0.01 mg/kg body weight to 20 mg/kg body weight,
   for example administered in divided doses up to four times a day.

An (ant)agonist of the present invention may be administered by any conventional route, for example enterally, e.g. including nasal, buccal, rectal, oral administration; parenterally, e.g. including intravenous, intraarterial, intramuscular, intracardiac, subcutanous, intraosseous infusion, transdermal (diffusion through the intact skin), transmucosal (diffusion through a mucous membrane), inhalational administration; topically; e.g. including epicutaneous, intranasal, intratracheal administration; intraperitoneal (infusion or injection into the peritoneal cavity); epidural (peridural) (injection or infusion into the epidural space); intrathecal (injection or infusion into the cerebrospinal fluid); intravitreal (administration via the eye); or via medical devices, e.g. for local delivery, e.g. stents;

e.g. in form of coated or uncoated tablets, capsules, (injectable) solutions, infusion solutions, solid solutions, suspensions, dispersions, solid dispersions; e.g. in the form of ampoules, vials, in the form of creams, gels, pastes, inhaler powder, foams, tinctures, lip sticks, drops, sprays, or in the form of suppositories.

For topical use, e.g. including administration to the eye, satisfactory results may be obtained with local administration of a 0.5-10%, such as 1-3% concentration of active substance several times daily, e.g. 2 to 5 times daily.

### DESCRIPTION OF THE FIGURES

FIG. 1

Subcellular Localization of GFP Tagged CerK WT and PH Domain-Mutant Proteins

COS-1 cells are transiently transfected with plasmids encoding CerK wild-type and mutant alleles N-terminally fused to GFP. 24 h after transfection, cells are analysed using fluorescent microscopy as described in the Methods section. Pictures shown are representative of the majority of the cellular population, observed in several experiments.

FIGS. 2 and 3

FIG. 2—In Vitro Activity Assay of CerK PH-Domain Mutants

Different mutant alleles of CerK are expressed in COS cells and assayed in vitro as crude cell lysates, in comparison to wild-type CerK (activity normalized to 100%). Data represent a mean of at least 2 experiments (-SD), performed in triplicates.

FIG. 3—In Cell Activity of CERK PH-Domain Mutants

In-cell kinase assay is performed for N-terminally GFP tagged WT CerK and key mutants, using ³²Pᵢ labeling followed by lipid extraction (upper panel, densitometric measurements as % relative to WT-CerK are indicated underneath). Lysates from control transfections are taken for Western Blot analysis (lower panel) using an antibody against the GFP tag.

FIGS. 4 and 5

FIG. 4—SDS-PAGE Running Behavior of the β6-β7 Loop Mutant Expressed in COS-1 Cells

N-terminally GFP tagged CerK and mutant alleles are overexpressed in COS-1 cells. SDS-PAGE is performed on the lysate and followed by Western Blot analysis using an antibody against the GFP tag. The lower non specific band (ns) is also present in a mock control (not shown).

FIG. 5—SDS-PAGE Running Behavior of the β6-β7 Loop Mutants Expressed In Vitro

In vitro translated ³⁵S Methionine labeled CerK WT and mutants are analysed by SDS-PAGE followed by autoradiography

FIGS. 6 and 7

β6-β7 Loop Mutants are Destabilized Proteins

FIG. 6—The β6-β7 Loop Mutants Display Increased Thermolability

Left, Whole cell lysates of COS-1 cells overexpressing WT or the CerK 90/91 mutant C-terminally FLAG-6×His tagged proteins are pre-incubated at 30° C. for the indicated times, before assaying for the remaining activity. Right, The presence of 20% glycerol prevents the inactivation of the 90/91 CerK mutant protein. This is one of two experiments with similar results.

FIG. 7—The β6-β7 Loop Mutants are More Sensitive to Trypsin

Wild type and mutant CerK, both C-terminally 6×His-FLAG tagged are overexpressed in COS-1 cells and harvested in lysis buffer. Lysates are incubated with different amounts of trypsin (0; 0.001; 0.01; 1 μg/ml). After a 30-min time incubation, samples are processed for PAGE analysis. Western blotting was performed using an anti-FLAG antibody. This is one of two experiments with similar results.

FIG. 8

The β6-β7 Loop Mutants are Prone to Aggregation

Western Blotting analysis performed as in FIG. 8. The open arrow heads indicate multimeric forms of the 90/91/96/98 mutant.

FIG. 9

β6-β7 Loop Mutants are not Recovered from Triton Soluble Membrane Compartments

Cell fractionation of wild type CerK compared to a CerK allele lacking the PH domain, to the 90/91/96/98 mutant of this study as well as to the kinase-dead CerK G198D mutant. All constructs are 6×His-FLAG tagged at the C-terminus. Cell fractionation is performed as described in the Methods section and obtained fractions are resolved by SDS-PAGE followed by Western Blot using an anti-FLAG antibody.

SEQUENCE LISTING

SEQ ID NO:1

(polynucleotide)
tgtgtaaagagagcacgacggcaccgctggaagtgggcgcag

SEQ ID NO:2

(polypeptide)
CVKRARRHRWKWAQ

SEQ ID NO:3

(polynucleotide Δ2-7)
gggcgacgggggccatggagccgctgcaatcc;

SEQ ID NO:4

(polynucleotide Δ2-13)
gagccgctgcaatccatggtgtgggtgaagcagc

SEQ ID NO:5

(polynucleotide; K17A)
ccgtgctgtgggtggcacagcagcgctgcgcc

SEQ ID NO:6

(polynucleotide R20A)
gtgggtgaagcagcaggcctgcgccgtgagcctg

SEQ ID NO:7

(polynucleotide K17A, R20A)
gtgggtggcacagcaggcctgcgcc

SEQ ID NO:8

(polynucleotide K33A)
gcgggctctgctggcctggtggcggagccc

SEQ ID NO:9

(polynucleotide R36A)
ctgctgcgctggtgggcgagcccggggccc

SEQ ID NO:10

(polynucleotide R29, 33, 36A)
gcggccgctctgctggcctggtgggcgagcccggggccc

SEQ ID NO:11

(polynucleotide, K74A)
catcaaggcagtggagcatggcagaaaatggaaaagc

SEQ ID NO:12

(polynucleotide K68V, K74A)
gaaacagacgttcacggggtgcatcaaggcagtggaaaatg

SEQ ID NO:13

(polynucleotide K77A)
cagtggaaaatggcaggcaatggaaaagccttacg

SEQ ID NO:14

(polynucleotide K80V)
ggcagaaaatggaagtgccttacgcttttacag

SEQ ID NO:15

(polynucleotide K74, 77A)
cagtggagcatggcaggcaatggaaaagc

SEQ ID NO:16

(polynucleotide K77, 80A)

SEQ ID NO:17

(polynucleotide)
ggcaggcaatggaagtgccttacgcttttacag

SEQ ID NO:18

(polynucleotide K90V)
gcttttacagttcactgtgtagtgagagcacgacggcac

SEQ ID NO:19

(polynucleotide R91A)
cagttcactgtgtaaaggcagcacgacggcaccg

SEQ ID NO:20

(polynucleotide K90V, R91A)
gcttttacagttcactgtgtagtggcagcacgacggcaccg

SEQ ID NO:21

(polynucleotide R93, 94A)
ctgtgtaaagagagcagcagtgcaccgctggaagtg

SEQ ID NO:22

(polynucleotide R96A)
gagagcacgacggcacgcctggaagtgggcgc

SEQ ID NO:23

(polynucleotide K98V)
gacggcaccgctgggtgtgggcgcaggtgac;

SEQ ID NO:24

(polynucleotide R96A, K98V)

SEQ ID NO:25

(polynucleotide)
gagagcacgacggcacgcctgggtgtgggcgcaggtgac

SEQ ID NO:26

(polynucleotide K90V, R91A, R96A, K98V)
acgacggcacgcctgggtgtgggcgcaggtgac;

SEQ ID NO:27

(polynucleotide L116G)
gctgtgtcacttgtgggggcagaccctgcgg

SEQ ID NO:28

(polynucleotide L119G)
cttgtggctgcagaccgggcgggagatgctgg

SEQ ID NO:29

(polynucleotide R120P)
ggctgcagaccctgcccgagatgctggagaagc

SEQ ID NO:30

(polypeptide)
KRARRHRWKW

In the following examples all temperatures are in degree Celsius. The following abbreviations are used

a.a. amino acid
CerK ceramide kinase
C1P ceramide-1-phosphate
ESP electrostatic potential
FCS fetal calf serum

### HMM Hidden Markov Model

GFP green-fluorescent protein
PAGE polyacrylamide gel electrophoresis
PBS phosphate-buffered saline
PDB Protein Structure Database; PIK, PtdIns kinase
PtdIns phosphatidylinositol
TLC thin-layer chromatography
WT wild-type

### EXPERIMENTAL PROCEDURES

### Materials

C8-ceramide is obtained from Cayman, cardiolipin from Sigma, octyl-D-beta-glucopyranoside from Fluka. [gamma-³²P]ATP (10 mCi/ml, 3000 Ci/mmol), [³²P]orthophosphate (10 mCi/ml) and [³⁵S]methionine are from Amersham Biosciences. Trypsin sequencing-grade and Complete™ protease inhibitors tablets are from Roche Molecular Biochemicals. Mutagenesis is performed with the QuickChange Site Directed Mutagenesis II Kit (Stratagene) and SDS-PAGE is done under reducing conditions on NuPAGE polyacrylamide gels (Invitrogen). NBD-labeled C6-ceramide is obtained from Molecular Probes. All other reagents are from Sigma, unless otherwise stated. Plasmid vectors as well as TOP10 competent E. coli cells are from Invitrogen. Oligonucleotide synthesis and DNA sequencing are performed at VBC-genomics.

### Human CerK Constructs

CerK cDNA, corresponding to Genebank™ accession number AB079066, is obtained and subcloned in Gateway™ compatible entry vectors e.g. as described in Billich, A., Bornancin, F., Devay, P., Mechtcheriakova, D., Urtz, N., and Baumruker, T, J. Biol. Chem. 278, 47408-47415, (2003) or Carre, A., Graf, C., Stora, S., Mechtcheriakova, D., Csonga, R., Urtz, N., Billich, A., Baumruker, T., and Bornancin, F., Biochem. Biophys. Res. Commun. 324, 1215-1219, (2004)). These plasmids are used for mutagenesis. Site directed mutagenesis is performed using the following primers (only the forward primers are indicated, changed bases are underlined). Combinations of mutants are performed using the same primers on already established mutants.

Δ2-7:
gggcgacgggggccatggagccgctgcaatcc;
Δ2-13:
gagccgctgcaatccatggtgtgggtgaagcagc;
K17A:
ccgtgctgtgggtggcacagcagcgctgcgcc;
R20A:
gtgggtgaagcagcaggcctgcgccgtgagcctg;
K17A, R20A:
gtgggtggcacagcaggcctgcgcc;
K33:
gcgggctctgctggcctggtggcggagccc;
R36A:
ctgctgcgctggtgggcgagcccggggccc;
R29, 33, 36A:
gcggccgctctgctggcctggtgggcgagcccggggccc;
K74A:
catcaaggcagtggagcatggcagaaaatggaaaagc;
K68V, K74A:
gaaacagacgttcacggggtgcatcaaggcagtggaaaatg;
K77A:
cagtggaaaatggcaggcaatggaaaagccttacg;
K80V:
ggcagaaaatggaagtgccttacgcttttacag;
K74, 77A:
cagtggagcatggcaggcaatggaaaagc;
K77, 80A:
ggcaggcaatggaagtgccttacgcttttacag;
K90V:
gcttttacagttcactgtgtagtgagagcacgacggcac;
R91A:
cagttcactgtgtaaaggcagcacgacggcaccg;
K90V, R91A:
gcttttacagttcactgtgtagtggcagcacgacggcaccg;
R93, 94A:
ctgtgtaaagagagcagcagtgcaccgctggaagtg;
R96A:
gagagcacgacggcacgcctggaagtgggcgc;
K98V:
gacggcaccgctgggtgtgggcgcaggtgac;
R96A, K98V:
gagagcacgacggcacgcctgggtgtgggcgcaggtgac;
K90V, R91A, R96A, K98V:
cacgacggcacgcctgggtgtgggcgcaggtgac;
L116G:
gctgtgtcacttgtgggggcagaccctgcgg;
L119G:
cttgtggctgcagaccgggcgggagatgctgg;
R120P:
ggctgcagaccctgcccgagatgctggagaagc

All constructs are transferred to pcDNA6.2DEST in order to express untagged proteins or pcDNA-DEST53 in order to express N-terminal GFP-fusion constructs.

### Overexpression of Wild-Type (wt) CerK and Mutant Proteins

COS-1 cells are obtained from DSMZ and cultured in DMEM/10% FCS at 37° C./5% CO₂ in a humidified atmosphere. Cells are seeded at 10⁵ cells/well in 6-well plates. After 24 hr, cells are transfected with 3 μg vectors containing expression constructs for different CerK alleles, using FuGENE 6 (Roche Molecular Biochemicals). Cells are incubated for 24 to 48 hr following transfection. For harvest, cells are washed with ice-cold washing buffer (lysis buffer but without Triton X-100 and Complete Protease Inhibitor), and scraped into lysis buffer (10 mM MOPS pH7.2; 2 mM EGTA, 150 mM KCl, 2% Triton X-100, 1 mM DTT and protease inhibitors. The suspension obtained is homogenized by 20 strokes in a Potter-Elvejhem homogenizer. Aliquots for kinase activity assay are used directly. Aliquots for Western Blotting analysis are processed as described below. In vitro translation is performed using the TNT coupled reticulocyte lysate system (Promega) as already described (see e.g. Carre, A., Graf, C., Stora, S., Mechtcheriakova, D., Csonga, R., Urtz, N., Billich, A., Baumruker, T., and Bornancin, F., ib.).

Assays of kinase activity—Ceramide kinase activity was measured in crude cell lysates according to published protocols as already described (see e.g. Carre, A., Graf, C., Stora, S., Mechtcheriakova, D., Csonga, R., Urtz, N., Billich, A., Baumruker, T., and Bornancin, F., ib.).). In-cell assays using either ³²Pᵢ or NBD-C6-ceramide followed by lipid extraction and analysis on TLC, are conducted as described previously (see e.g. Bornancin, F., Mechtcheriakova, D., Stora, S., Graf, C., Wlachos, A., Devay, P., Urtz, N., Baumruker, T., and Billich, A., Biochim. Biophys. Acta 1687, 31-43 (2005)).

### Tryptic Proteolysis

Trypsin is dissolved in 1 mM HCl to 10 μg/ml and diluted in lysis buffer (see above) to 0.004 μg/ml; 0.04 μg/ml and 4 μg/ml. COS-1 cell lysates are mixed 3:1 with trypsin dilutions, to achieve final trypsin concentrations of 0.001; 0.01 and 1 μg/ml. Samples are incubated for 30 minutes at 30° C., and then processed for Western Blotting as described below.

### Cell Fractionation

Lysis is performed in 500 μl of buffer 1 (20 mM Tris pH 7.5; 1 mM DTT). After homogenization, lysates are centrifuged at 55,000 rpm in a TLA120.2 rotor using a TL-100 Ultracentrifuge (Beckman). Supernatants correspond to fraction 1. Pellets are dissolved in 500 μl of buffer 2 (20 mM Tris pH 7.5; DTT 1 mM; NaCl 250 mM) and samples are spun as before. Supernatants (fraction 2) are stored and pellets dissolved in 500 μl of buffer 3 (Tris pH 7.5 20 mM; DTT 1 mM; Triton X-100 1%). Samples are spun again and final supernatants collected (fraction 3). Final pellets are resuspended in 500 μl of buffer 1 and stored (fraction 4).

### Protein Quantification

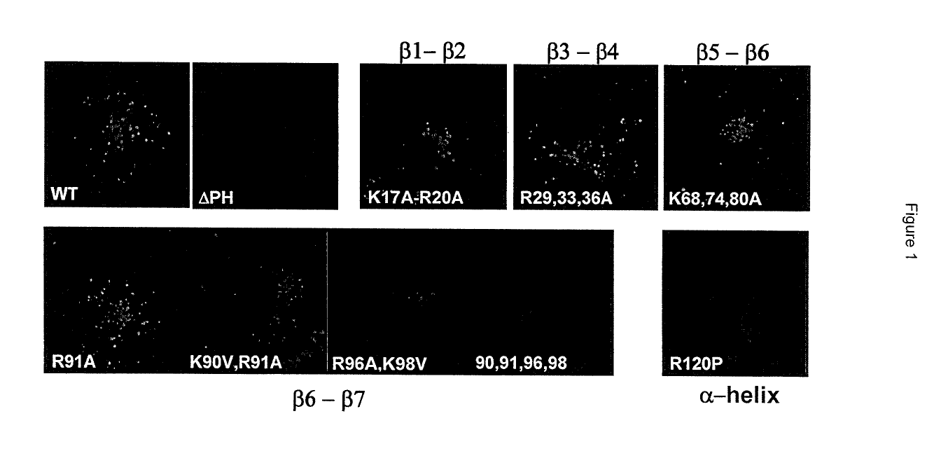

10 μl of whole cell lysate are diluted in 1 ml of EtOH and incubated on dry ice for 15 minutes. After centrifugation (15 minutes at maximum speed in a benchtop centrifuge) the supernatant is discarded and 10 μl of 0.1 N NaOH 0.1 N and 200 μl of micro BCA (Pierce) working solution are added. Following vigorous vortexing and incubation at 37° C. for 45 min 100 μl are read on a Molecular Device SpectraMax 340 PC 384 plate reader at 562 nm. BSA is used as the standard. Normalisation for CerK protein content used for FIG. 1 and Table 1 is obtained by measuring GFP fluorescence of the lysates with a Molecular Devices SpectraMax Gemini XS (Ex/em: 395/507 nm; cut-off: 495 nm).

### Western Blot Analysis

All fractions are diluted in 3:1 in 4× NuPAGE LDS sample buffer supplemented with 200 mM of DTT, and incubated at 75° C. for 10 min before snap freezing and storage at −80° C. PAGE analysis is carried out with NuPAGE Bis-Tris 4-12% run in MOPS buffer at 115 V for 2 hr. Gels are transferred to Hybond-ECL nitrocellulose membranes (Amersham Biosciences) in a BioRad Trans-Blot SD Semi-Dry Transfer cell (25V and 100 mA per membrane, for 2 hr) and probed with an anti-GFP antibody (Abcam ab290 rabbit polyclonal) followed by an anti-rabbit Ig antibody linked to horseradish peroxidase (# NA9340V, Amersham Biosciences), or with an anti-FLAG antibody (Sigma, M2) followed by an anti-mouse Ig antibody linked to horseradish peroxidase (# NA9310V, Amersham Biosciences). Detection is carried out with ECL (Amersham Biosciences) or Lumiglo (Cell Signalling) using either Hyperfilms ECL (Amersham Biosciences) or a Fujifilm Intelligent Dark Box LAS 3000 imager. Band intensities are measured using ImageJ 1.33u (Wayne Rasband NIH http://rsb.info.nih.gov/ij/Java 1.3.1_{—}03).

### Fluorescence Microscopy

COS-1 cells are seeded into 4-chambered coverslips (LabTEK II-Nalge Nunc) at 2.2×10⁴ cells per chamber. 24 hours after seeding cells are transfected with 0.6 μg plasmid per chamber using FuGENE 6. Live-cell fluorescence microscopy is done 24 h after transfection on an inverted microscope Axiovert 200 M equipped with a high resolution microscopy camera AxioCam MRc (Zeiss) and objectives Plan-Neofluar 40×/1.30 oil DIC and Plan-Apochromat 63×/1.40 oil DIC. The filter system is suited for detection of both wild-type GFP and EGFP (Ex 470/40 nm, BS 495 nm, Em 525/50 nm).

### Results

### Subcellular Localization and Activity of CerK PH Domain Mutants

It was previously reported that recombinant CerK constitutively localizes to the Golgi complex and cytoplasmic vesicles, in both COS-1 cells or primary HUVEC (see e.g. Carre, A., Graf, C., Stora, S., Mechtcheriakova, D., Csonga, R., Urtz, N., Billich, A., Baumruker, T., and Bornancin, F., ib.). This localization pattern is completely lost when the PH domain is removed; ΔPH-CerK instead shows diffuse cytoplasmic compartmentalization HUVEC (see e.g. Carre, A., Graf, C., Stora, S., Mechtcheriakova, D., Csonga, R., Urtz, N., Billich, A., Baumruker, T., and Bornancin, F., ib.). This clear readout is used to characterize the CerK PH domain mutant proteins of the present study. As can be seen in FIG. 1, localization of GFP-CerK proteins is almost unchanged upon mutagenesis of positively charged residues of either the β1-β2 loop (17/20: K17A and R20A), the β3-β4 loop (29/33/36: R29,33,36A) or the β5-β6 loop (68/74/80: K68,74,80A). In contrast, mutagenesis of the β6-β7 loop impaired the ability of CerK to localize to the Golgi complex, either partially (90/91: K90A,R91A and 96/98: R96A,K98V) or totally when these two double mutations were combined (90/91/96/98). The latter mutant localized in the cell as would ΔPH-CerK. When the C-terminal alpha-helix is compromised (R120P), the resulting mutant protein becomes similarly mislocalized (FIG. 1)

These results identify the β6-β7 loop loop as the key provider of positively charged amino acids to allow for localization of the wild-type protein. A minor role for the β1-β2 as well as β3-β4 loop becomes visible with mutants displaying a partially compromised β6-β7 loop (Table 1). Instead, positive charges within the long β5-β6 loop do not appear to be involved, since their substitution does not aggravate the phenotype of such partially compromised mutants (Table 1).

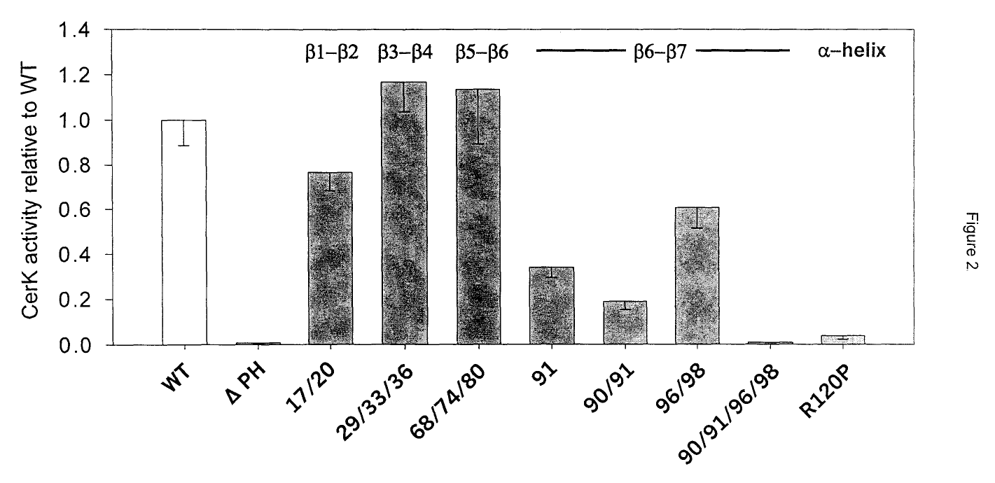

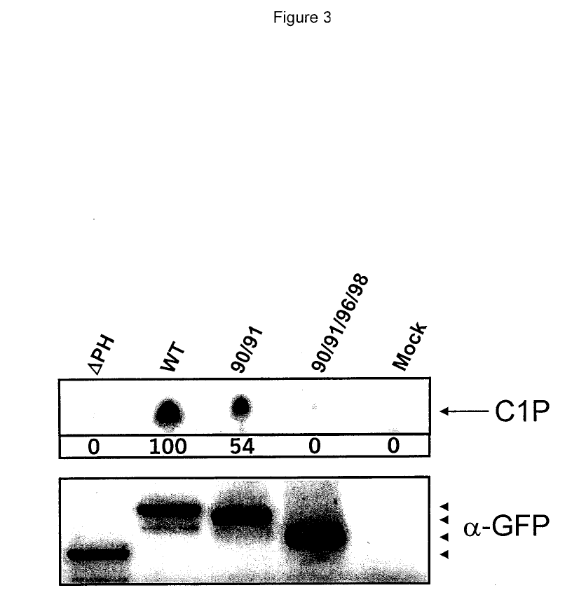

CerK activity is measured in vitro for the above mutants (FIG. 2, Table 1). All mutants displaying wild-type localization also displays wild-type catalytic activity (29/33/36, 68/74/80); those with partial loss of localization have lower activity (e.g. 90/91, 90/98); finally the mutants with the most aberrant localization show the weakest activity (90/91/96/98, R120P). Therefore it seems that loss of localization parallels loss of activity. Cell-based CerK assays are performed to test compromised mutants more directly. Consistent with the in vitro assay, the 90/91 mutant shows partial activity whereas the 90/91/96/98 is totally devoid of activity (FIG. 3).

Hence, the PH domain of CerK influences enzymatic activity through a mechanism that does not involve substrates recognition.

β6-β7 Loop Compromised Mutants are Full-Length Proteins with Increased SDS-PAGE Mobility

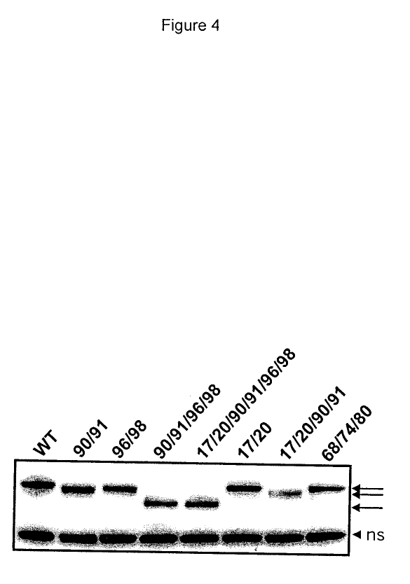

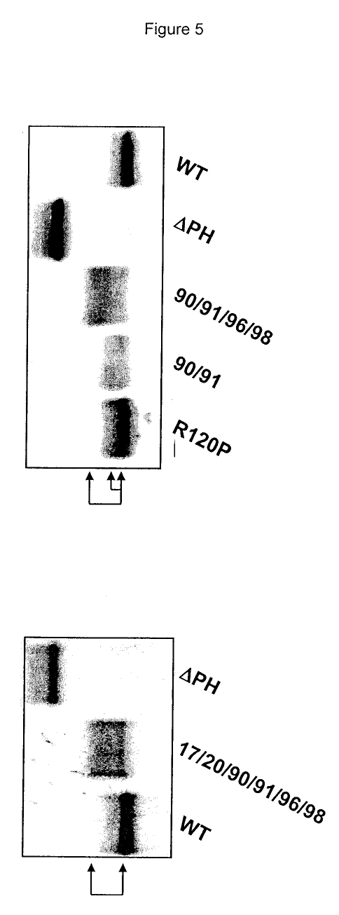

As seen in FIG. 3, he 90/91/96/98 mutant displays a strikingly different running behavior on SDS-PAGE gels, about halfway between wild-type and ΔPH CerK. A survey among the variety of mutants made for this study is made, to ask whether the running pattern seen for the 90/91/96/98 mutant is shared by others. As seen in FIG. 4, mutation of amino acids 90 and 91 induces a small downwards mobility shift, not seen with any other mutations including those at residues 96 and 98 (cf Table 1). However, mutations at residues 96 and 98 synergize with those at residues 90 and 91, giving raise to the large downward shift mentioned. Therefore, a fast running behavior typifies mutants with a compromised β6-β7 loop. Further evidence for modified running behavior is obtained upon in vitro translation of untagged proteins in vitro. In vitro translated β6-β7 loop mutants showed a smeary pattern, displaying fast running protein material as well as nearly wild-type-like running material in variable amounts (FIG. 5). This is an indication that the mobility shifts observed β6-β7 loop mutants are not a mere consequence of the net loss of positive charges due to mutagenesis is unlikely to be a mechanism for enhanced migration of β6-β7 loop mutants. Given the large change in observed running behavior as well as diffused appearance of the detected β6-β7 loop mutants proteins, next it is examined whether glycosylation could have been counteracted in the β6-β7 loop mutants proteins, thus leading to the observed fast running species. However, not any glycosylation in either wild-type or 90/91/96/98 mutant proteins (not shown) are detected. Thus these experiments establish that the shift in migration observed for the β6-β7 loop mutants is not due to proteolysis or lack of a modification such as glycosylation.

### CerK PH Domain β6-β7 Loop Features

The experiments above suggest that the β6-β7 loop displays important structural features. Mutagenesis of this loop has actually resulted in changes that are still visible after denaturation, i.e. the mutant protein shows enhanced globularity and thus, reduced apparent molecular size. The β6-β7 loop of CerK PH domain ranks among the longest β6-β7 loops when compared to PH domains of known structure. Of note, this loop is highly charged and would display two additional strands, β6′ and β6″, linked by a motif of intercalated positively charged and hydrophobic residues (KRARRHRWKW). The presence of tryptophans in this region is unique compared to all PH domains of known structure. Using the program Modeler, 100 possible β6-β7 loop conformations (data not shown) are sampled. According to secondary structure analysis and manual inspection of the resulting loops, no favored conformation can be derived from the produced sampling. One may, however, propose at least two functions for the above mentioned motif:

(i) hydrophobic residues may serve as an anchor towards the core of the protein to help position the charged residues;
(ii) alternatively, they may be used as buffers between charged residues.

### β6-β7 Loop Mutants Display a Destabilized Conformation

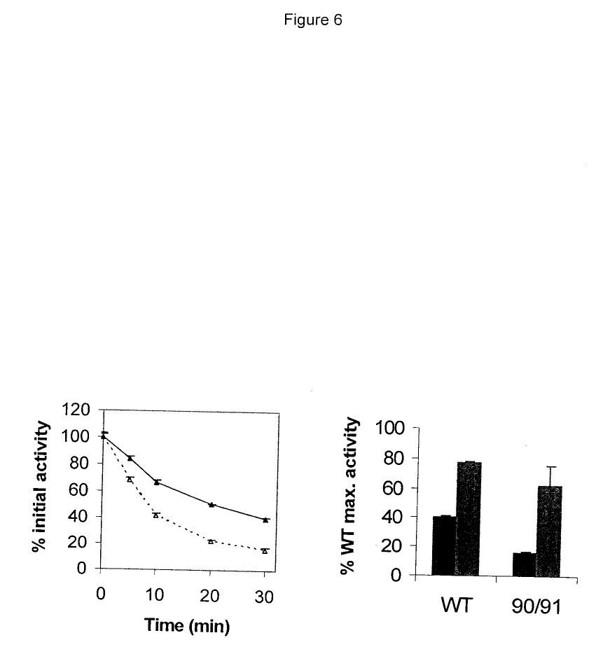

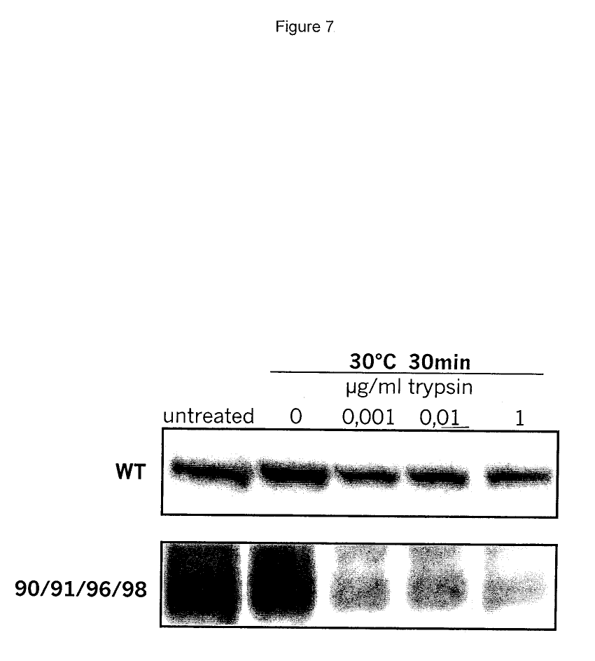

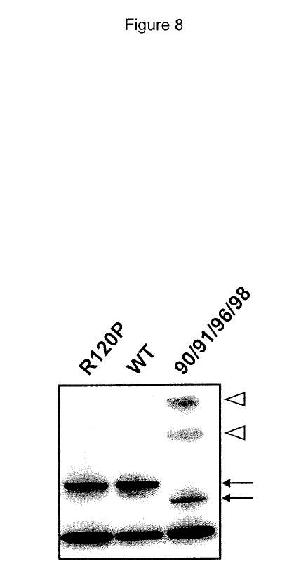

The half life of CerK enzyme activity is reduced two-fold when comparing the 90/91 mutant to the wild-type protein (FIG. 6). Indeed, 20 min at 30° C. decreases the activity of the wild-type protein by 50%, whereas a similar reduction occurs in less than 10 min for the mutant. Accordingly, the inclusion of glycerol significantly slows the inactivation process, allowing the mutant to approach wild-type activity levels (FIG. 6, right). Therefore, the PH domain appears to allow for conformational stabilization and the β6-β7 loop plays a role in this process. This is also exemplified when looking at the trypsin sensitivity of the 90/91/96/98 mutant compared to the wild-type protein. As shown in FIG. 7 trypsin concentrations as low as 0.001 μg/ml result in almost complete disappearance of full length 90/91/96/98 mutant protein whereas wild-type CerK remains undegraded when incubated with 1 μg/ml trypsin for the same period of time. Furthermore, its observed that β6-β7 loop mutants are prone to aggregation as seen from the frequent detection of high molecular weight multimeric bands following SDS-PAGE analysis (FIG. 8). This indicates that absence of positively charged residues in this loop has exposed sites within the protein leading to multimer formation. Exposure of Ala, Val, Cys and Trp residues in the β6-β7 loop itself might be able to induce multimer formation through an increased hydrophobic surface. Aggregation is absent in the R120P mutant, which is destabilized within the C-terminal loop but has an intact β6-β7 loop (FIG. 8). Overall, mutants with a compromised β6-β7 loop display an unstable conformation which is subject to enhanced deactivation, proteolysis, and aggregation.

β6-β7 Loop Mutants are not Recovered from Triton-Soluble Membrane Compartments

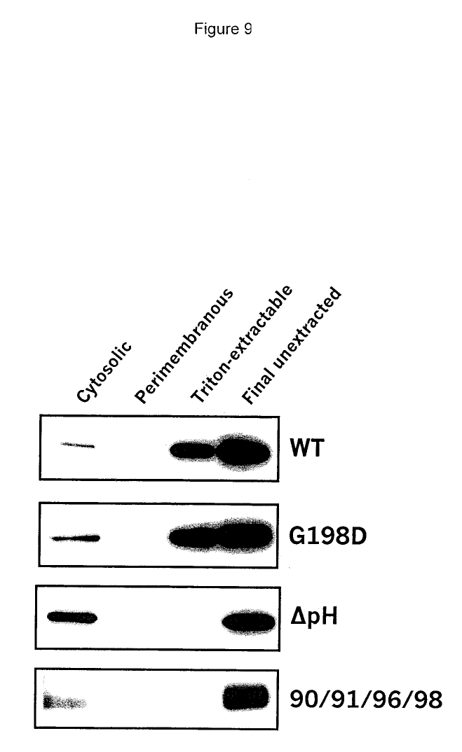

Recombinant wild-type CerK mostly associates with the particulate fraction when expressed in HEK293 (see e.g. Sugiura, M. et al, J. Biol. Chem. 277, 23294-23300, (2002)) and COS-1 cells (see e.g. Carre, A., Graf, C., Stora, S., Mechtcheriakova, D., Csonga, R., Urtz, N., Billich, A., Baumruker, T., and Bornancin, F. Biochem. Biophys. Res. Commun. 324, 1215-1219, (2004)). It is found that dissociation of the particulate protein requires prolonged treatment with a combination of detergent, salt and chelators (FIG. 7A). This is consistent with early reports on solubilization of ceramide kinase activity from brain membranes (see e.g. Bajjalieh, S., and Batchelor, R. Methods Enzymol. 311, 207-215, (2000)). Still, some CerK protein can readily be harvested from the cytosolic and membrane fractions (FIG. 9). In a similar way, the G198D catalytically inactive mutant of CerK, which possesses a functional PH domain and localizes as the wild-type enzyme when fused to GFP (not shown), also displays some cytosolic and membrane extractable material (FIG. 9). In contrast, neither the CerK ΔPH nor the CerK 90/91/96/98 proteins are recovered from the membrane extractable fraction. This confirms the requirement for the PH domain to associate with membranes—as previously shown using in vitro translated proteins and liposomes (see e.g. Carre, A., Graf, C., Stora, S., Mechtcheriakova, D., Csonga, R., Urtz, N., Billich, A., Baumruker, T., and Bornancin, ib.)—and demonstrates the critical role played by the β6-β7 loop in this process. Importantly, in the absence of a functional PH domain, mutant proteins are still largely present at the particulate fraction.

### CerK Mutants

The CerK mutants are categorized according to the mutagenized region. Localization is depicted using a 3 black-dotted scale: (•••) wild-type, (••∘) partially compromised, (•∘∘) severely compromised, (∘∘∘) ΔPH CerK-type localisation. Activity is expressed as a percentage of that of WT-CerK, after normalization of CerK protein amounts. Data represent the mean+/−SD of at least two experiments, performed in triplicates. The phenotype of the apparent molecular weight as seen from the SDS-PAGE running behavior is indicated in the right most column. Deletion of the first 7 amino acid residues of CerK (Δ2-7 CerK) has no effect on the phenotype of the observed protein, consistent with the PH domain starting at residue number 8 (Carre, A., Graf, C., Stora, S., Mechtcheriakova, D., Csonga, R., Urtz, N., Billich, A., Baumruker, T., and Bornancin, F. Biochem. Biophys. Res. Commun. 324, 1215-1219, (2004), FIG. 2). Further deletion (Δ2-13 CerK) removes most of the first β-strand and completely abrogates wild type localization and activity. Such a strong phenotype can also be obtained with a W15D CerK mutant (not shown). An already described L10A mutation (see Kim, T. J. et al FEBS Lett. 579, 4383-4388, (2005)) is partially effective in the assays.

TABLE 1
Summary of CerK mutant proteins and associated phenotypes

| | CERK construct | Localisation | Activity (% +/− SD) | App. MW |
|---|---|---|---|---|
| | WT | ••• | 100 | wt |
| | ΔPH | ∘∘∘ | <1 | n.a. |
| PH domain region | Δ2-7 | ••• | 60 +/− 18 | wt |
| | Δ2-13 | ∘∘∘ | <1 | wt |
| β1-β2 | K17A | ••• | — | — |
| | R20A | ••• | — | — |
| | K17A, R20A | ••∘ | 77 +/− 8 | wt |
| β2-β3 | R29A, R33A | ••• | — | — |
| | K33A | ••• | — | — |
| β3-β4 | R36A | ••• | — | — |
| | R29, 33, 36A | ••• | 117 +/− 13 | wt |
| β5-β6 | K74A | ••• | — | — |
| | K68V, K74A | ••• | — | — |
| | K77A | ••• | — | — |
| | K80V | ••• | — | — |
| | K74, 77A | ••• | — | wt |
| | K77, 80A | ••• | 102 +/− 35 | wt |
| | K 68, 74, K80V | ••• | 114 +/− 24 | wt |
| β6-β7 | K90V | ••• | 92 +/− 16 | wt |
| | R91A | ••∘ | 34 +/− 5 | wt |
| | K90V, R91A | •∘∘ | 19 +/− 4 | 90/91 |
| | R93, 94A | ••• | — | — |
| | R96A | ••• | 103 +/− 26 | — |
| | K98V | ••• | 120 +/− 14 | — |
| | R96A, K98V | •∘∘ | 61 +/− 9 | wt |
| | K90V, R91A, R96A, K98V | ∘∘∘ | <1 | 90/91/96/98 |
| β1-β2 + β6-β7 | K17A, R20A, K90V, R91A | ∘∘∘ | 6 +/− 3 | 90/91 |
| | K17A, R20A, K90V, R91A, R96A, K98V | ∘∘∘ | <1 | 90/91/96/98 |
| β3-β4 + β6-β7 | R29, 33, 36 A, K90V, R91A | ∘∘∘ | 2 +/− 1 | 90/91 |
| β5-β6 + β6-β7 | K77, 80A, K90V, R91A | •∘∘ | 18 +/− 8 | 90/91 |
| α-helix | L116G | ••• | — | wt |
| | L119G | ∘∘∘ | — | wt |
| | R120P | ∘∘∘ | 4 +/− 2 | wt |
| | Δ118-124 | ∘∘∘ | — | — |

1. An isolated polynucleotide encoding a polypeptide of SEQ ID NO:2.

2. The isolated polynucleotide of claim 1, wherein said polynucleotide has the sequence set forth in SEQ ID NO:1.

3. An isolated polypeptide of SEQ ID NO: 2.

4. An isolated polypeptide encoded by a polynucleotide of SEQ ID NO:1.

5. A vector comprising a polynucleotide of claim 1.

6. An expression system comprising a polynucleotide of claim 1, wherein said expression system or part thereof is capable of producing a polypeptide of SEQ ID NO:2.

7. A host cell comprising an expression system according to claim 6.

8. A method for producing an isolated polypeptide of SEQ ID NO:2, comprising culturing a host cell according to claim 7 under conditions sufficient for the production of said polypeptide in the culture and recovering said polypeptide from the culture.

9. A method for producing a recombinant host cell which produces a polypeptide SEQ ID NO:2 comprising transforming or transfecting a host cell with the expression system according to claim 6, such that the host cell, under appropriate culture conditions, produces said polypeptide.

10. (canceled)

11. A screening assay for identifying an agonist or an antagonist of ceramide kinase, wherein said assay comprises:
a) a polypeptide according to claim 3, or
b) a recombinant cell expressing a polypeptide of claim 3, or
c) a cell membrane expressing a polypeptide of claim 3, and
d) means for determining a binding effect of a candidate compound with a polypeptide according to claim 3.

12. A method for identfying an agonist or antagonist of ceramide kinase, which method comprises
A) contacting
A1) a polypeptide according to claim 3, or
A2) a recombinant cell expressing a polypeptide of claim 3, or
A3) a cell membrane expressing a polypeptide of claim 3, with a candidate compound;
B) determining a binding effect of a candidate compound with the polypeptide of any of A1), A2) or A3),
C) determining the effect of said candidate compound on production or biological activity of;
C1) ceramide kinase protein, or
C2) a recombinant cell expressing ceramide kinase protein, or
C3) a cell membrane expressing ceramide kinase protein, or
C4) an antibody to ceramide kinase protein; and
D) choosing an agonist or antagonist determined in step C).
